(19) United States
(12) Patent Application Publication
(10) Pub. No.: US 2024/0420020 A1
(43) Pub. Date: Dec. 19, 2024

(54) PARTIAL ORDER REDUCTION TO INCREASE PLANNER SPEED

(71) Applicant: INTERNATIONAL BUSINESS MACHINES CORPORATION, Armonk, NY (US)

(72) Inventors: Michael KATZ, Goldens Bridge, NY (US); Junkyu LEE, San Diego, CA (US)

(21) Appl. No.: 18/342,814

(22) Filed: Jun. 28, 2023

Related U.S. Application Data
(60) Provisional application No. 63/508,494, filed on Jun. 15, 2023.

(51) Int. Cl. G06N 20/00

(52) U.S. Cl. CPC G06N 20/00

(57) ABSTRACT

Computer implemented methods, systems, and computer program products include program code executing on a processor(s) that obtains a planning problem and one or more bounding conditions for the solutions. The program code transforms the planning problem into a single-goal form. The program code computes stubborn sets over the single-goal form of the planning problem. The program code defines a pruned search space utilizing the single-goal form of the planning problem and the stubborn sets. The program code performs a K* search over the pruned search space. The program code obtains the set of solutions.

### CROSS-REFERENCE TO RELATED APPLICATION

This application claims priority to U.S. Provisional Application No. 63/508,494, filed Jun. 15, 2023, entitled “PARTIAL ORDER REDUCTION TO INCREASE PLANNER SPEED,” which is incorporated herein by reference in its entirety, for all purposes.

### BACKGROUND

The present invention relates generally to the field of artificial intelligence (AI) in computing and specifically, reducing central processing unit (CPU) time to process instructions that problem solving in top-quality planning.

Artificial intelligence (AI) refers to intelligence exhibited by machines. Artificial intelligence (AI) research includes search and mathematical optimization, neural networks, and probability. Artificial intelligence (AI) solutions involve features derived from research in a variety of different science and technology disciplines ranging from computer science, mathematics, psychology, linguistics, statistics, and neuroscience. Machine learning has been described as the field of study that gives computers the ability to learn without being explicitly programmed.

Automated planning and scheduling, referred to as AI planning is a branch of AI that concerns the realization of strategies or action sequences, typically for execution by intelligent agents, autonomous robots, and/or unmanned vehicles. Because solutions in AI planning are more complex than classical control and classification problems, AI planning problems are generally discovered and optimized in multidimensional space. AI planning, also referred to as automated planning, aims to solve problems, modeled in an input language, that involve finding a strategy of action provided they are modeled in a suitable input language. Optimal planning in AI planning refers to finding one best solution to a problem. A planning problem in AI refers to a problem with some initial starting state, which one wishes to transform into a desired goal state through the application of a set of actions.

### SUMMARY

Shortcomings of the prior art are overcome, and additional advantages are provided through the provision of a computer-implemented method for generating a set of solutions for a planning problem. The computer-implemented method can include: obtaining, by one or more processors, a planning problem; obtaining, by the one or more processors, one or more bounding conditions for each solution in the set of solutions for the planning problem; based on the one or more bounding conditions, transforming, by the one or more processors, the planning problem into a single-goal form of the planning problem; computing, by the one or more processors, stubborn sets over the single-goal form of the planning problem; defining, by the one or more processors, a pruned search space utilizing the single-goal form of the planning problem and the stubborn sets; performing, by the one or more processors, a K* search over the pruned search space; and obtaining, by the one or more processors, based on the K* search, the set of solutions.

Shortcomings of the prior art are overcome, and additional advantages are provided through the provision of a computer program product for generating a set of solutions for a planning problem. The computer program product comprises a storage medium readable by a one or more processors and storing instructions for execution by the one or more processors for performing a method. The method includes, for instance: obtaining, by the one or more processors, a planning problem; obtaining, by the one or more processors, one or more bounding conditions for each solution in the set of solutions for the planning problem; based on the one or more bounding conditions, transforming, by the one or more processors, the planning problem into a single-goal form of the planning problem; computing, by the one or more processors, stubborn sets over the single-goal form of the planning problem; defining, by the one or more processors, a pruned search space utilizing the single-goal form of the planning problem and the stubborn sets; performing, by the one or more processors, a K* search over the pruned search space; and obtaining, by the one or more processors, based on the K* search, the set of solutions.

Shortcomings of the prior art are overcome, and additional advantages are provided through the provision of a system for generating a set of solutions for a planning problem. The system includes: a memory, one or more processors in communication with the memory, and program instructions executable by the one or more processors via the memory to perform a method. The method includes, for instance: obtaining, by the one or more processors, a planning problem; obtaining, by the one or more processors, one or more bounding conditions for each solution in the set of solutions for the planning problem; based on the one or more bounding conditions, transforming, by the one or more processors, the planning problem into a single-goal form of the planning problem; computing, by the one or more processors, stubborn sets over the single-goal form of the planning problem; defining, by the one or more processors, a pruned search space utilizing the single-goal form of the planning problem and the stubborn sets; performing, by the one or more processors, a K* search over the pruned search space; and obtaining, by the one or more processors, based on the K* search, the set of solutions.

Computer systems and computer program products relating to one or more aspects are also described and may be claimed herein. Further, services relating to one or more aspects are also described and may be claimed herein.

Additional features and advantages are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 depicts one example of a computing environment to perform, include and/or use one or more aspects of the present invention;

FIG. 2 is a workflow that provides an overview of various aspects performed by the program code (executing on one or more processors) in some embodiments of the present invention;

FIG. 3 is a flow chart that provides an overview of various aspects performed by the program code (executing on one or more processors) in some embodiments of the present invention; and

FIG. 4 is a workflow that provides an overview of various aspects performed by the program code (executing on one or more processors) in some embodiments of the present invention.

### DETAILED DESCRIPTION

Automated planning or AI planning is a sub-area of AI that includes solving problems where the solution involves finding a strategy of action. Program code, executed by at least one processor, solves these problems, in part, by the program code modeling the problems in an effective input language. This automated planning includes finding one best solution to a problem. Although various optimal planners exist and can be used to solve certain very specific types of problems, when dealing with diverse problems of a high complexity, including PSPACE-hard problems, there is no present approach that can handle the breadth of problems of this complexity. PSPACE (polynomial space) refers to a set of all decision problems that can be solved by a Turing machine using a polynomial amount of space. A problem considered PSPACE-hard if an algorithm that solves the problem can be transformed into an algorithm that solves any other problem in PSPACE in a polynomial amount of time. The term PSPACE-hard is used to designate a complexity class in theoretical computer science that captures the set of decision problems that can be solved by a deterministic Turing machine using a polynomial amount of memory space in which the problems are unlikely to have efficient solutions.

An example of a type of problem that various AI planning approaches can be used to attempt to solve is unordered top-quality planning. This problem can be articulated as given q, the program code tries to find all plans of cost up to q times optimal. In solving this problem, the program code may skip re-orderings of the plans. Re-orderings can be skipped because, from an application perspective, often two valid plans that are re-orderings of each other are equivalent. Thus, if the program code were to provide both, it would be providing duplicative information and would waste processing time and resources in generating and providing both plans. Existing AI planning approaches to solving unordered top-planning problems include, but are not limited to, a symbolic search based approach (i.e., program code provides top-quality plans and checks duplicates based on unordered variants), a forbid iterative based approach (i.e., program code utilizes cost-optimal planners to find a single plan and iteratively reformulates a planning problem to reduce a space of plans), and a K* search based approach (i.e., program code utilizes a two-phase search to iteratively develop a search space and attempts to extract top-quality solutions, which includes the program code finding all top-quality plans and checking duplicates based on unordered variants). A drawback shared by these approaches, as presently utilized, is that they are time and resource intensive. Thus, there is a need to increase the speed at which AI planners operate without increasing the processing resources to accommodate this change in speed.

Embodiments of the present invention are inextricably tied to computing and are directed to a practical application. AI planning is a branch of AI, which is inextricably tied to computing. However, the unordered top-quality planning problems that are solved by aspects of embodiments of the present invention also enable improvements to the technical computing field. As mentioned earlier, the examples herein increase the speed at which a processor can derive solutions to unordered top-quality planning problems, thus reducing CPU time. Additionally, these unordered top-quality planning problems are specific challenges in computing, including but not limited to, hypothesis exploration for malware detection in computing systems, scenario planning for enterprise risk management, state projection, and automated large-scale data analysis. Plan recognition through AI planning can detect malware because the program code (executing on one or more processors) can detect malware based on the program code deriving potentially unreliable observations from network traffic. In a healthcare setting, AI planning can assist care providers in early detection of health complications in an intensive care unit setting. There are examples of AI planning being utilized in the energy domain, including to project the price of oil and volume of oil produced into the future (e.g., fifteen years into the future). In the aforementioned risk management setting, AI planning can be utilized to assist financial organizations in identifying and managing emerging risks. Thus, aspects of the examples herein can be integrated into systems that solve various challenges that are unique to computing and given that deriving these solutions is inextricably tied to computing, aspects herein improve the functioning of the computing system processing the unordered top-quality planning problem by reducing CPU time for deriving a solution. The examples herein are directed to a practical application (and improved computing) at least because the examples herein are directed to computing a set of top-quality plans using partial order reduction, increasing the speed of generating top-quality plans and faster generation of top-quality plans is crucial for the products and services that depend on planning as their computational engines. The example herein, when integrated into certain existing solutions, can speed up the computation of plans, scale better on larger problems in the future, and scale to return more plans that are different from the application perspective. In some examples, integrating aspects of the approaches discussed herein into finding solutions for top-quality planning problems can increase (task) coverage by approximately 50% and can reach maximal coverage much faster.

Examples herein include computer-implemented methods, computer system, and computer program products that include program code executing on one or more processor that find top-quality solutions for planning problems. When compared to other approaches, in the examples herein provide significant improvements over existing approaches at least because in the examples herein, the program code can operate more quickly (without requiring additional processing resources to increase the speed of computation) by including partial order reduction in the automated planning performed by the program code. Examples herein provide a significant improvement to a K* based top-quality planner (e.g., an AI planner that utilizes the K* search based approach) through partial order reduction. This partial order reduction is achieved, in part, by utilizing aspects of a A* search and Eppstein's algorithm (EA). An A* search approach and/or algorithm is a popular technique used in path-finding and graph traversal such that program code comprising various games and web-based maps utilize an A* algorithm to approximate a shortest path very efficiently. When applying an A* algorithm, the program code would consider a square grid having many obstacles and as input, would obtain a starting cell and a target cell. The program code would apply the A* algorithm to determine how to reach the target cell (if possible) from the starting cell as quickly as possible. By applying the A* algorithm, the program code selects, at each step, the node according to a value, “f”, which is a parameter equal to the sum of two other parameters, “g” and “h”. At each step, the program code selects a node/cell having a lowest “f”, and processes that node/cell. The parameter “g” is the movement cost to move from a starting point to a given square on the grid, following the path generated to get there. The parameter “h” is the estimated movement cost to move from that given square on the grid to the final destination and considered a heuristic approach (e.g., proceeding to a solution by trial and error or by rules that are only loosely defined). The program code, applying the A* algorithm cannot determine an actual distance until it computes the path because of environmental factors that could impact the route. Meanwhile, program code can utilize EA to determine a shortest path as well as all possible deviations from this shortest path. For example, program code applying EA can enumerate, in order of increasing length, the number of shortest paths between a given pair of nodes in a weighted digraph G with n nodes and m arcs. To solve this problem using EA, the program code computes a shortest path tree and then builds a graph D (G) representing all possible deviations from the shortest path. Once the program code generates the graph, it can obtain the shortest paths can be obtained in order of increasing length.

The program code in some of the examples herein utilizes a K* based top-quality planner by interleaving A* search EA to extract top solutions from an explicit graph (A* search space). A* searches are formulated in terms of weighted graphs; the program code executing the search starts from a specific starting node of a graph and utilizes the A* search to find a path to the given goal node having the smallest cost (e.g., least distance travelled, shortest time, etc.). In the examples herein, if the program code executing the K* based top-quality planner (interleaving A* search EA) does not extract enough (based on a pre-determined criteria) solutions, the program code continues to execute the A* search until switching criteria triggers. In case of (unordered) top-quality planning, no switching is performed until the f-layer that corresponds to the cost bound is exhausted. The advantage of partial order reduction in the examples herein are realized when the program code improves the A* search performance for finding a first, optimal plan with the help of partial order reduction. When the program code performs the A* search, the program code can prune successors of a node are pruned in a way that preserves at least some re-ordering of existing plans. This successor pruning techniques include stubborn sets (e.g., an optimality-preserving pruning technique for factored state-space search including classical planning with applicability limited by computational overhead). In the examples herein, if the successor function in an A* iteration of K* is pruned with stubborn sets, then the program code, utilizing the (enhanced) K* based top-quality planner described herein, will generate all plans except those that are re-orderings of found plans. Thus, in the examples herein, program code executing on one or more processors, can safely use stubborn sets-based pruning in a K* planner to solve unordered top-quality planning problems.

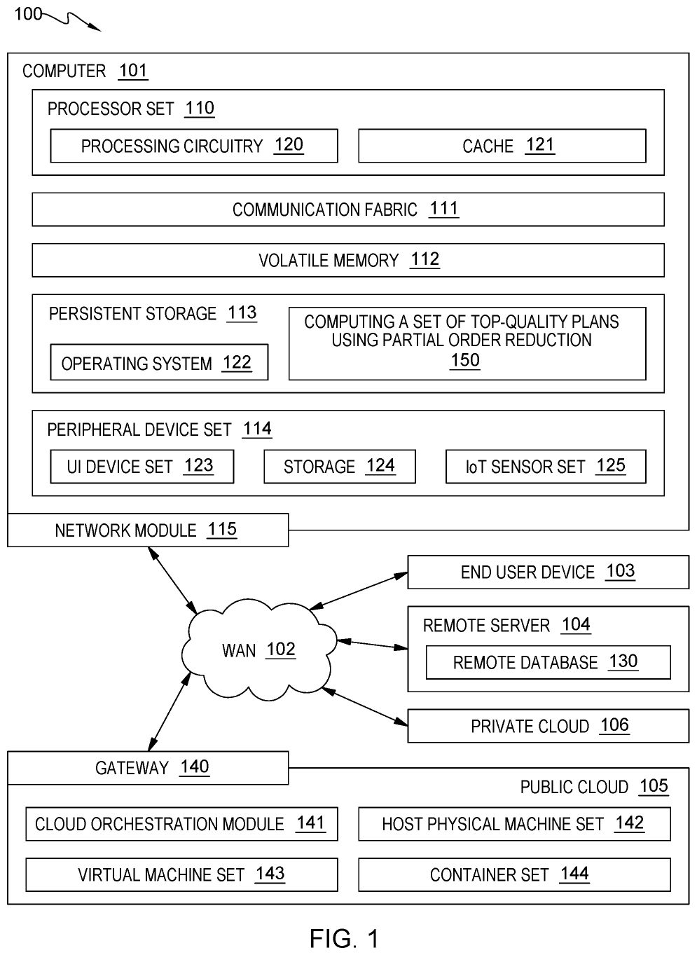

One example of a computing environment to perform, incorporate and/or use one or more aspects of the present disclosure is described with reference to FIG. 1. In one example, a computing environment 100 contains an example of an environment for the execution of at least some of the computer code involved in performing the inventive methods, such as a code block for computing a set of top-quality plans using partial order reduction 150. In addition to block 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 150, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 150 in persistent storage 113.

Communication fabric 111 is the signal conduction path that allow the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in block 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation and/or review to an end user, this recommendation would typically be communicated from network module 115 of computer 101 through WAN 102 to EUD 103. In this way, EUD 103 can display, or otherwise present, the recommendation and/or review to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation and/or review based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

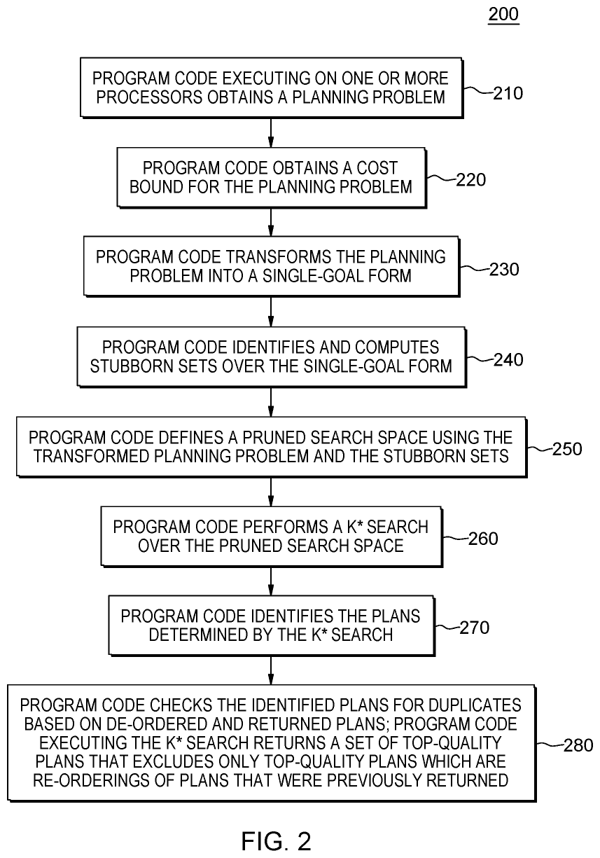

FIG. 2 is a workflow 200 that illustrates various aspects of some embodiments of the present invention. Examples herein comprise program code executing on one or more processors that computes a set of top-quality plans using partial order reduction. In the example illustrated in the workflow 200 of FIG. 2, program code executing on one or more processors obtains a planning problem (210). A planning problem in AI is a problem with some initial starting state, which the program code can transform into a desired goal state by applying a set of actions. The program code obtains a cost bound for the planning problem (220). Based on the cost bound and the planning problem, the program code transforms the planning problem into a single-goal form (230). A cost bound is an expense or expenditure for acquiring, producing, or maintaining something, usually measured in money, time, or energy outlay. In AI and computing a cost bound can refer to processing resources. Limiting the goals for an AI planning problem, in view of cost considerations can aid in generating the set of top-quality plans.

A search problem, which includes planning problems, can be characterized by factors including, but not limited to, an initial state, a set of possible actions and/or applicability conditions, a goal, and a path cost function. The initial state is the state at the beginning of the search. In the set of possible actions, an action is only applicable to a subset of the world states, which can generally be reduced to a single state. When one can reduce the subset of states into a single state, the actions come together with applicability conditions, to know for which states they are applicable. A goal can be represented by either a single goal state or a set of goal states. One way to solve a search problem (e.g., planning problem) is to start with a path starting from an initial state, navigate through one or more intermediate states using only applicable actions, and end at a goal state. The path cost function attributes a numerical value to a path, its cost. A lower numerical value is preferable because it translates to a lower cost (e.g., lower processing time, fewer resources, etc.). In embodiments of the present invention, the program code transform an initial planning problem such that there is a single-goal state, e.g., a single goal to attain via the solutions to the planning problem.

The program code identifies and computes stubborn sets over the single-goal form (240). A stubborn set method is known as a partial order methods. Partial order methods are based on a selective search algorithm. In a stubborn set method, at each state processed during the search, the program code calculates a stubborn set is calculated and used enabled transitions of this set to generate the successors of the state. To calculate stubborn sets, the program code detects dependencies between transitions in terms of conflict and causality. Various known approaches for computing stubborn states can be utilized by the program code to calculate the stubborn sets. Utilizing stubborn sets will enable the program code to prune permutable parts of forward search in planning. One challenge with an A* search is that it stores all generated nodes in memory. Thus, by generating stubborn sets, the program code can prune the nodes and control the processing utilized by the program code in determining a plan. Hence, in examples herein, the program code defines a pruned search space using the transformed planning problem and the stubborn sets (250). The program code performs a K* search over the pruned search space (260). In some examples, the program code performs the K* search interleaving an A* search and EA to extract top solutions from an explicit graph including the A* search space. The K* search interleaving an A* search and EA can include the program code continuing the A* search until switching criteria triggers in response to not extracting a threshold number of solutions. This threshold can be pre-defined, for example, based on Additionally, the for unordered top-quality planning, the program code will not perform switching until an f-layer that corresponds to the cost bound is exhausted. In some examples, the A* search identifies a first optimal plan and is improved using the partial order reduction. This partial order reduction occurs because the program code prunes successors of a node and the program code allows for preserving at least some reordering of existing plans. In this examples, successor pruning techniques include the stubborn sets. The program code identifies the plans determined by the K* search (270). The program code checks the identified plans for duplicates based on de-ordered and returned plans (280). Thus, in some examples, the program code executing the K* search returns a set of top-quality plans that excludes only top-quality plans which are re-orderings of plans that were previously returned (already in the set). In some examples, the program code can automatically implement one or more of the returned plans.

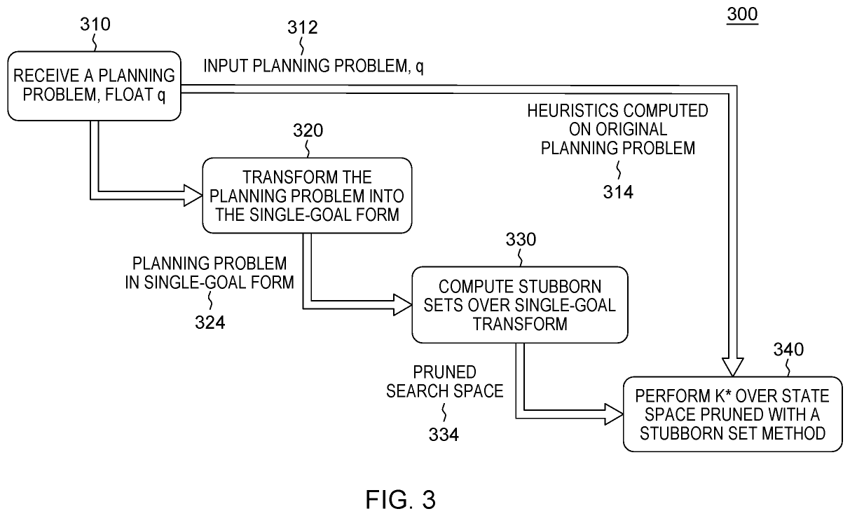

FIG. 3 is a flowchart 300 that also illustrates various aspects of some examples herein. This flowchart 300 highlights the combination of a heuristic search and an orthogonal that combine to return a set of top quality plans. As illustrated in FIG. 3, the program code obtains a planning problem “q” (310). In some embodiments of the present invention, the inputs (e.g., planning problem, q). In some examples, the program code can generate the inputs (planning problems), the planning problems, to include metadata and/or data to indicate to the program code generating the plans that the process for generating these plans should include partial order reduction based pruning. The program code provides the planning problem (the input) to (312) to a heuristic approach (e.g., A* search with or without EA) and utilizing the heuristic approach the program code computes heuristics on the original planning problem (e.g., q) (340). Examples of heuristics include but are not limited to, bidirectional search, A* search, simulated annealing, hill climbing, best first search, and beam search. Symmetrically or asymmetrically with computing the heuristics on the original planning problem (e.g., the input, q), the program code transforms the planning problem into a single-goal form (320). The program code provides the single-goal form of the planning problem 324 to program code (which can be another module, or the same module, depending on the configuration). The program code utilizes the single-goal form of the planning problems to generate stubborn sets, generating a pruned search space 334 (330). The program code (which can be a separate module or the same module) obtains the heuristics computed on the original planning problem and the pruned search space and performs a K* search over the pruned space with a stubborn set method to generate a set of top-quality plans (340). Because of the program code pruning the space with the stubborn sets, the set does not include top-quality plans which are re-orderings of plans already in the set. In some examples, the program code can automatically implement one or more of the returned plans.

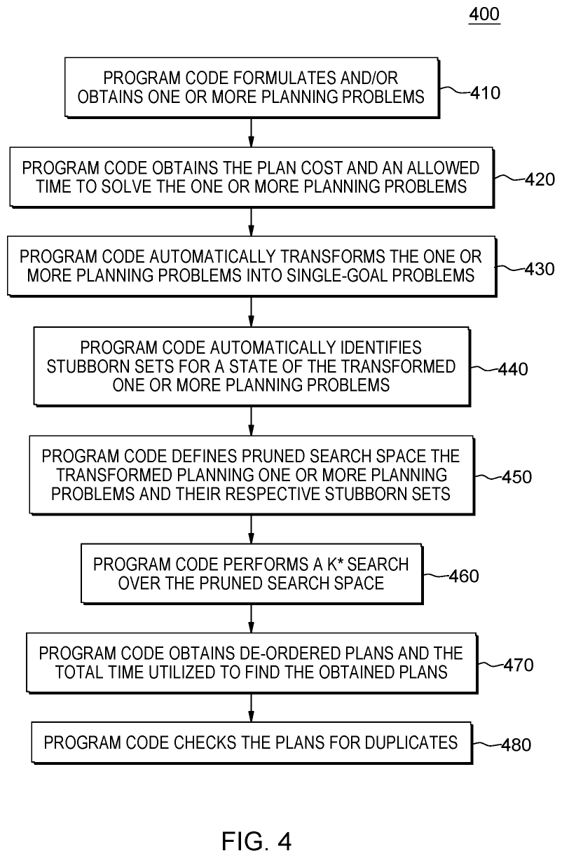

FIG. 4 is a workflow 400 that illustrates aspects of some examples herein. In both FIGS. 2 and 4, when the program code computes top-quality plans, it does so, based in part on bounds that are taken as input by the program code (executing on one or more processors). These bounds assist the program code in transforming the planning problem into a single-goal planning problem. Thus, the program code can utilize both an initial state and a goal to determine a plan for navigation (e.g., a top quality plan). Thus, the program code in some examples computes or generates a set of top-quality plans by formulating and/or obtaining one or more planning problems (410). The program code obtains the plan cost and an allowed time to solve the one or more planning problems (420). The program code automatically transforms the one or more planning problems into single-goal problems (430). The program code automatically identifies stubborn sets for a state of the transformed one or more planning problems (440). The program code defines pruned search space the transformed planning one or more planning problems and their respective stubborn sets (450). The program code performs a K* search over the pruned search space (460). Responsive to executing the K* search, the program code obtains de-ordered plans and the total time utilized to find the obtained plans (470). The program code checks the plans for duplicates (480). Based on the program code identifying any duplicates, the set of top quality plans does not include top-quality plans which are re-orderings of plans already in the set. In some examples, the program code can automatically implement one or more of the returned plans.

Embodiments of the present invention include computer-implemented methods, computer program products, and computer systems that comprise program code executing on one or more processors that generates a set of solutions for a planning problem. The program code obtains a planning problem. The program code obtains one or more bounding conditions for each solution in the set of solutions for the planning problem. Based on the one or more bounding conditions, the program code transforms the planning problem into a single-goal form of the planning problem. The program code computes stubborn sets over the single-goal form of the planning problem. The program code defines a pruned search space utilizing the single-goal form of the planning problem and the stubborn sets. The program code performs a K* search over the pruned search space. The program code obtains, based on the K* search, the set of solutions. A technical advantage of the described computer-implemented methods, computer program products, and computer systems is that implementing the aspects described increases the speed of generating top-quality plans and faster generation of top-quality plans is crucial for the products and services that depend on planning as their computational engines, many of which are discussed herein.

Various additional examples of the computer-implemented methods, computer program products, and computer systems are described below, and these examples, including and excluding the additional examples enumerated below, in any combination (provided these combinations are not inconsistent), increase the speed of generating top-quality plans.

In some examples, each solution comprises a top-quality plan addressing the planning problem.

In some examples, the set of solutions does not include solutions that comprise re-orderings of other solutions in the set.

In some examples, the program code generates the planning problem.

In some examples, the bounding conditions include one or more of: solution cost and time to solve the planning problem.

In some examples, obtaining the set of solutions comprises obtaining a total time value for generating the set of solutions.

In some examples, the program code checks the set of solutions for duplicates. The program code can also remove the duplicates.

In some examples, each top-quality plan comprises tasks.

In some examples, the program code automatically implements, in a computing system, at least one solution of the set of solutions.

In some examples, the program code generates heuristics on the planning problem.

The program code can utilize the heuristics to perform the K* search over the pruned search space.

In some examples, the program code generating the heuristics includes the program code interleaving, an A* search and Eppstein's algorithm (EA) to extract the heuristics from an explicit graph including an A* search space, wherein the heuristics comprise top solutions for the explicit graph.

In some examples, the program code interleaving the A* search and EA includes the program code extracting, graph solutions. The program code can determine that the graph solutions do not comprise a threshold number of graph solutions. Based on the program code determining that the graph solutions do not comprise the threshold number of graph solutions, the program code continues the A* search until meeting a pre-defined switching criteria.

In some examples, the program code terminates the A* search based on exhausting a layer corresponding to a bounding condition of the one or more bounding conditions.

In some examples, the bounding condition is a cost bounding.

In some examples, the program code generating the heuristics on the planning problem includes the program code determining a first optimal solution. The program code utilizes the heuristics to perform the K* search over the pruned search space. The K* search comprises the program code utilizing the stubborn sets to prune successor nodes to the first optimal solution to generate the set of solutions.

Although various embodiments are described above, these are only examples. For example, reference architectures of many disciplines may be considered, as well as other knowledge-based types of code repositories, etc., may be considered. Many variations are possible.

Various aspects and embodiments are described herein. Further, many variations are possible without departing from a spirit of aspects of the present invention. It should be noted that, unless otherwise inconsistent, each aspect or feature described and/or claimed herein, and variants thereof, may be combinable with any other aspect or feature.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method for generating a set of solutions for a planning problem comprising:
obtaining, by one or more processors, a planning problem;
obtaining, by the one or more processors, one or more bounding conditions for each solution in the set of solutions for the planning problem;
based on the one or more bounding conditions, transforming, by the one or more processors, the planning problem into a single-goal form of the planning problem;
computing, by the one or more processors, stubborn sets over the single-goal form of the planning problem;
defining, by the one or more processors, a pruned search space utilizing the single-goal form of the planning problem and the stubborn sets;
performing, by the one or more processors, a K* search over the pruned search space; and
obtaining, by the one or more processors, based on the K* search, the set of solutions.

2. The method of claim 1, wherein each solution comprises a top-quality plan addressing the planning problem.

3. The method of claim 1, wherein the set of solutions does not include solutions that comprise re-orderings of other solutions in the set.

4. The method of claim 1, further comprising:
generating, by the one or more processors, the planning problem.

5. The method of claim 1, where the bounding conditions include one or more of: solution cost and time to solve the planning problem.

6. The method of claim 1, wherein obtaining the set of solutions comprises obtaining a total time value for generating the set of solutions.

7. The method of claim 1, further comprising:
checking, by the one or more processors, the set of solutions for duplicates; and
removing, by the one or more processors, the duplicates.

8. The method of claim 2, wherein each top-quality plan comprises tasks.

9. The method of claim 1, further comprising:
automatically implementing, by the one or more processors, in a computing system, at least one solution of the set of solutions.

10. The method of claim 1, further comprising:
generating, by the one or more processors, heuristics on the planning problem; and
utilizing, by the one or more processors, the heuristics to perform the K* search over the pruned search space.

11. The method of claim 10, wherein generating the heuristics comprises:
interleaving, by the one or more processors, an A* search and Eppstein's algorithm (EA) to extract the heuristics from an explicit graph including an A* search space, wherein the heuristics comprise top solutions for the explicit graph.

12. The method of claim 11, wherein interleaving the A* search and EA comprises:
extracting, by the one or more processors, graph solutions;
determining, by the one or more processors, that the graph solutions do not comprise a threshold number of graph solutions; and
based on the determining, continuing the A* search until meeting a pre-defined switching criteria.

13. The method of claim 10, further comprising:
terminating, by the one or more processors, the A* search, based on exhausting a layer corresponding to a bounding condition of the one or more bounding conditions.

14. The method of claim 13, wherein the bounding condition is a cost bounding.

15. The method of claim wherein generating the heuristics on the planning problem comprises:
determining, by the one or more processors, a first optimal solution; and
utilizing, by the one or more processors, the heuristics to perform the K* search over the pruned search space, wherein the K* search comprises utilizing the stubborn sets to prune successor nodes to the first optimal solution to generate the set of solutions.

16. A computer system comprising:
a memory; and
one or more processors in communication with the memory, wherein the computer system is configured to perform a method, said method comprising:
obtaining, by the one or more processors, a planning problem;
obtaining, by the one or more processors, one or more bounding conditions for each solution in the set of solutions for the planning problem;
based on the one or more bounding conditions, transforming, by the one or more processors, the planning problem into a single-goal form of the planning problem;
computing, by the one or more processors, stubborn sets over the single-goal form of the planning problem;
defining, by the one or more processors, a pruned search space utilizing the single-goal form of the planning problem and the stubborn sets;
performing, by the one or more processors, a K* search over the pruned search space; and
obtaining, by the one or more processors, based on the K* search, the set of solutions.

17. The computer system of claim 16, wherein each solution comprises a top-quality plan addressing the planning problem.

18. The computer system of claim 16, wherein the set of solutions does not include solutions that comprise re-orderings of other solutions in the set.

19. A computer program product for generating a set of solutions for a planning problem comprising:
a computer readable storage media having program instruction embodied therewith, the program instructions executable by a processing circuit, to cause the processing circuit to:
obtain, by processing circuit, a planning problem;
obtain, by the processing circuit, one or more bounding conditions for each solution in the set of solutions for the planning problem;
based on the one or more bounding conditions, transform, by the processing circuit, the planning problem into a single-goal form of the planning problem;
compute, by the processing circuit, stubborn sets over the single-goal form of the planning problem;
define, by the processing circuit, a pruned search space utilizing the single-goal form of the planning problem and the stubborn sets;
perform, by the processing circuit, a K* search over the pruned search space; and
obtaining, based on the K* search, by the processing circuit, the set of solutions.

20. The computer program product of claim 19, wherein each solution comprises a top-quality plan addressing the planning problem.
